(19) United States
(12) Patent Application Publication
(10) Pub. No.: US 2011/0300232 A1
(43) Pub. Date: Dec. 8, 2011

(54) EFFECTS OF NITRIC OXIDE (NO) AND iNOS ON EXPRESSION AND SIGNAL TRANSDUCTION IN CANCER AND ITS USE IN DRUG DISCOVERY AND CANCER THERAPY DESIGN

(76) Inventor: Pooncharas Tipgunlakant, Belmont, CA (US)

(21) Appl. No.: 13/155,370

(22) Filed: Jun. 7, 2011

Related U.S. Application Data
(60) Provisional application No. 61/352,816, filed on Jun. 8, 2010.

(51) Int. Cl. A61K 33/26; G01N 33/573; A61K 38/06; A61K 31/15; A61K 31/04; A61K 31/19; A61K 31/196; C12Q 1/26; A61P 35/00

(52) U.S. Cl. 424/608; 435/25; 435/7.4; 514/18.9; 514/742; 514/562; 514/565; 514/611

(57) ABSTRACT

Nitric Oxide (NO) acts as double-edged sword, which induces and prevents cell death, depending on various factors. The mechanism for the NO regulation of cells is not fully understood. The present invention provides experiment design methods and therapy design methods leveraging viable hypothesis supported by current research findings. This invention determines the effects of NO by controlling the majority parameters such as the steady-state concentration of NO, duration of NO exposure and the local level of oxygen. The experiment designs are structured to improve understanding of NO regulation of cells, and further can be directly adapted for therapy design. The invention directs these experiment design and therapy design methods to the study and treatment of cancer. In an application, the effects of exogenous NO on inducible Nitric Oxide Synthase (iNOS) expression and signal transduction in ovarian cancer is applied to drug discovery and therapy design for ovarian and other cancers.

### CROSS-REFERENCE TO RELATED APPLICATIONS

Pursuant to 35 U.S.C. §119(e), this application claims benefit of priority from Provisional U.S. patent application Ser. No. 61/352,816, filed Jun. 8, 2010, the contents of which are incorporated by reference.

### COPYRIGHT & TRADEMARK NOTICES

A portion of the disclosure of this patent document may contain material, which is subject to copyright protection. Certain marks referenced herein may be common law or registered trademarks of the applicant, the assignee or third parties affiliated or unaffiliated with the applicant or the assignee. Use of these marks is for providing an enabling disclosure by way of example and shall not be construed to exclusively limit the scope of the disclosed subject matter to material associated with such marks.

### BACKGROUND OF THE INVENTION

Nitric Oxide (NO) acts as a double-edged sword, both inducing and preventing cell death. To elucidate the mechanism controlling the balance between the NO induced protective and destructive action would be crucial to use in drug discovery and therapy design.

The occurrence of cancer is second only to heart disease in developed countries. Several advances have been made in treatment of various cancers such as chemotherapy, γ-irradiation, immunotherapy or suicide gene therapy. The effect of triggering apoptosis in cancer cells is primarily used in antitumor therapies [1][2].

Beside the novel therapeutic agents under laboratory and clinical trials, Nitric Oxide (NO) has been studied as endogenous NO and NO donors that play a role in NO-based cancer therapy (for example by increasing extensive DNA damage, oxidative/nitrosative stress, apoptosis, mitochondrial damage, and cytotoxicity. [3][4][5]). In contrast, Anti-NO approaches are used in cancer therapeutic strategies because, in its contrasting role, NO promotes cancer progression by increasing DNA mutation, invasiveness, angiogenesis, cytoprotection, immune tolerance and decreasing apoptosis [5].

For the apoptosis effect, NO inhibits the activity of NFkB, a major of anti-apoptosis pathway, and NO stimulate p53 pathway which response to DNA damage [9]. Also, NFkB and Akt induce nitric oxide synthase (NOS) to synthesize NO, regulated by the accumulation of p53 which inhibits NOS activity [6] (and thus synthesis of NO).

Consequently, on one hand, NO supports tumor regression and cell death. On the other hand, NO promotes cancer development and progression. Thus NO is a double-edged sword in interacting with the cell apoptosis mechanism. FIG. 1 provides a depiction of the double-edged sword effects of NO.

Although mechanisms of NO regulation are not fully understood, it is possible to predict many effects of NO by observing the effects of experimentally controlling dominant relevant parameters such as the steady-state concentration of NO, the duration of NO exposure, and the level of oxygen in the experimental circumstance. For example, the ability of NO to both induce and prevent cell death are not unlikely to be affected by its redox state, concentration, superoxide and other molecules. For example:
It has been well established that low level of NO promotes cell proliferation while high level of NO induces apoptosis.
Negative feedback of Nitric Oxide (NO) via the effects of inducible Nitric Oxide Synthase (iNOS) can be an important factor that regulates the concentration of NO in the cell. Therefore, negative feedback of NO might reduce the production of NO form iNOS, and a low concentration of NO will increase the lifetime of cell.
In addition, the studies of NO have variety of experimental methods that have uncontrolled parameters causing the different effects of NO in either different cell lines or same cell lines, such as the steady state of NO concentrations, range of the concentration of NO, time of NO exposure, and the circumstance of the experimental method. As the results, the controlling of steady state of NO, time of NO exposure and the oxygen in the environment should be of concern for the study the effect of NO to cancer.

Accordingly, even though the mechanisms, which determine the role of NO in the cell death and survival have not been thoroughly characterized, one can deduce that the effects of NO could depend on the concomitant of steady state concentrations, duration of NO exposure, and level of oxygen in the circumstance.

Consideration of the interaction of each apoptosis pathway from NO is essential in augmenting the cancer therapeutic efficiency and drug development. Thus a useful goal for cancer therapy is one leveraging the mechanisms controlling the balance between the NO related protective and destructive action in cancer cells.

Based on the above, a viable central hypothesis for use in a cancer therapy design approach is that the cummulative concentrations of NO and duration of NO exposure regulate iNOS expression and cell apoptosis. To explore and develop such approaches, an understanding of how NO regulates the proliferation of cancer cells is developed along with tactics to modify the methods of NO delivery for cancer therapy. In particular, tactics to modify the methods of NO delivery can utilize controlling the level of NO, time of NO exposure, and other circumstance of experiments or treatment.

It is noted that the effects of NO on apoptosis can be measured via cell proliferation colorimetric assays using enzyme processable dyes such as MMT (3-(4,5-Dimethylthiazol-2-yl-2,5-diphenyltetrazolium bromide, a yellow tetrazole), XTT, MTS, WSTs or other dyes that naturally exhibit a first color (such as the yellow of MTT) and reduce by living cells to a dye exhibiting a different color (such as the purple of formazan dyes). The effects of NO on iNOS expression can be measured via the Western blot method. Thus, one way to test key aspects of NO relevant design hypothesis (or processes related to aspects of the hypothesis) can utilize the following specific steps:

Step 1: Determine if level of NO and time of NO exposure regulate the expression of iNOS and apoptosis.

A first viable therapy and experiment design hypothesis is that the long-term exposure of cells to low concentration of exogenous nitric oxide causes negative feedback mechanism inducing down regulation of iNOS and promotes tumor progression. It is well known that low concentration of NO reduces apoptosis. Thus, low concentration of exogenous NO causes down-regulation of iNOS, which in turn results in the reduce ability of NO production from available iNOS. The lower level of NO resulting from this down-regulated iNOS will prolong the lifetime of cancer cells. To observe the effects of NO to the responses of iNOS, one can use Western blot method to determine the expression of iNOS. In addition, cellular survival can be assessed using a MTT assay. Accordingly, it is natural to expect that iNOS expression as measured by Western blot will display down-regulation and a low value of the inhibition rate (% IR) from an MTT assay by long period of low level of NO exposure.

A second viable therapy and experiment design hypothesis is that high concentration of exogenous NO does not affect to the expression of iNOS but rather causes the apoptosis of cells by other means. Most reports have shown that high concentrations of NO have a destructive effect. Thus, in spite of the beneficial effects of low NO concentration, it is to be expected that concentrations of NO high enough to cause cell apoptosis will not affect the negative feedback mechanism involving iNOS. Accordingly, Accordingly, it is natural to expect that the expression of iNOS from Western blot method will not indicate change after the cells are exposed to high level of exogenous NO, but this same level of concentration will on the other hand result in a high value of measured % RT from MTT assay.

STEP 2: Determine if the circumstance of the experiment or treatment has an effect on iNOS expression.

Another viable therapy and experiment design hypothesis is that low concentrations of NO cause down-regulation of iNOS under hypoxia more than in normoxia. Since high level of oxygen in the environment, normoxic condition increases metabolism of NO. However, low level of oxygen, hypoxic condition will maintain NO in the environment because it retards NO degradation. Therefore, it is to be expected that duration of NO exposure under hypoxia conditions will be longer than duration of NO exposure under normoxia conditions. Increasing the duration of NO exposure will cause down-regulation of iNOS by the associated and aforementioned negative-feedback effect. Accordingly, it is to be expected that the iNOS expression by using western blot method.

Researchers have demonstrated how NO plays role in apoptosis mechanism in cell by presenting the expression of iNOS as a negative feedback effect. The present invention is concerned with how variations in the level of NO and exposure duration affect iNOS expression in cancer cells. Understanding the effect of exogenous NO to apoptosis pathway will contribute to specification of potential carcinogenic therapies and efficient drug development.

### SUMMARY OF THE INVENTION

Features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

In an embodiment, inventive methods of the invention comprise effects of NO by controlling the steady-state concentration of NO, duration of NO exposure and the local level of oxygen.

In aspect of the invention, experimental procedures taught in the specification can be combined with candidate therapeutic agents that are designed to produce conditions matching those of particular situations that appear advantageous for control of cancer processes.

In an aspect of the invention, the invention directs these experiment design and therapy design methods to the study and treatment of cancer.

In an aspect of the invention, the effects of exogenous NO on iNOS expression and signal transduction in ovarian cancer is applied to drug discovery and therapy design for ovarian and other cancers.

Another aspect of the invention leverages the fact that the different effects of NO are caused from the different signal transduction and uncontrolled parameters such as the steady-state concentrations.

Another aspect of the invention leverages the fact that the concentration of NO and duration of NO exposure can be estimated by regulated HIF-α expression.

Another aspect of the invention leverages the expected relation that the concentration and duration of NO exposure plays role in apoptosis pathways.

Another aspect of the invention leverages the expected relation that these factors can potentially affect iNOS.

Another aspect of the invention leverages the expected relation that high concentration of NO does not affect to iNOS expression but instead promotes cellular apoptosis.

Another aspect of the invention leverages the expected relation that time of NO exposure under hypoxia will be longer than time of NO exposure under normoxia conditions.

Another aspect of the invention leverages the expected relation that increasing time of NO exposure will cause down-regulation of iNOS by negative feedback effect.

Another aspect of the invention leverages the expected relation that the factors that should be of concern are the concentration of NO, duration of NO exposure, and the circumstance of NO exposure.

Additionally, the invention provides for various aspects of the experiment data and experiment design to be used in creating a cancer therapy that manipulates exogenous NO in hypoxic areas of cancer cells.

In one approach, an agent comprised by the cancer therapy is used to replace at least one NO donor in at least one portion of at least one of the procedures described.

In another approach, an agent comprised by the cancer therapy comprises at least one NO donor.

In an approach to creating the cancer therapy, an agent comprised by the cancer therapy controls the level of NO concentration.

In an approach to creating the cancer therapy, an agent comprised by the cancer therapy controls the duration of NO exposure.

Additionally, the invention provides for use of the aforedescribed methods to create a cancer therapy. The experimental methods and measurements produced by them are used to identify ranges of NO concentrations and NO exposure durations which cause apoptosis of cancer cells residing in a hypoxic environment. Using this, one or more agents are identified that can produce these ranges of NO concentrations and NO exposure durations. The therapy would then comprise deploying the agent into a hypoxic area comprised by cancer cells wherein the agent manipulates the NO concentration and NO exposure duration within said ranges, resulting in apoptosis of the cancer cells.

In an example embodiment or implementation of such a cancer therapy, the agent comprises at least one NO donor.

In an embodiment, the invention provides a method for designing experiments for the study of Nitric Oxide (NO) in the control of cell death for use in cancer therapies, the method comprising:
establishing that long term exposure of cells to low concentration of exogenous nitric oxide causes a negative feedback mechanism to induce down-regulation of inducible nitric oxide synthase (iNOS) by choosing a first NO donor that can produce a relatively low level of NO concentration over a relatively long NO-production half-life, applying that NO donor to a cell culture media at selected time points, measuring the levels of iNOS concentration and NO concentration to produce first measurements, and recording first data corresponding to the first measurements;
establishing that high concentration of exogenous NO does not affect to the expression of iNOS but instead will cause apoptosis of cells by choosing a second NO donor that can produce a relatively high level of NO concentration over a relatively short NO-production half-life, applying that NO donor to a cell culture media at selected time points, measuring the levels of iNOS and NO concentration to produce second measurements, and recording second data corresponding to the second measurements; and
establishing that for a given level of NO concentration, the expression of iNOS down-regulates more in hypoxic conditions than in normal oxygen conditions by choosing a third NO donor that generates a relatively low concentration of NO, applying that NO donor to a cell culture media at selected time points, measuring the levels of iNOS and NO concentration to produce third measurements, recording third data corresponding to the third measurements, and comparing the iNOS response between normal and hypoxic environments,
   wherein at least one of the first data, second data, and third data are used to create a cancer therapy that manipulates exogenous NO in a hypoxic area of cancer cells.

In an embodiment, the invention provides a method for designing a cancer therapy by manipulating the concentration and duration of Nitric Oxide (NO) for the control of cell death, the method comprising:
using a first NO donor to produce a relatively low level of NO concentration over a relatively long NO-production half-life, applying that NO donor to a cell culture media at selected time points, measuring the levels of iNOS and NO concentration to produce first measurements, and recording first data corresponding to the first measurements;
using a second NO donor to produce a relatively high level of NO concentration over a relatively short NO-production half-life, applying that NO donor to a cell culture media at selected time points, measuring the levels of iNOS and NO concentration to produce second measurements, and recording second data corresponding to the second measurements; and
using a third NO donor to produce a relatively low concentration of NO, applying that NO donor to a cell culture media at selected time points, measuring the levels of iNOS and NO concentration to produce third measurements, recording third data corresponding to the third measurements, and comparing the iNOS response between normal and hypoxic environments,
   wherein at least one of the first data, second data, and third data are used to create a cancer therapy that manipulates exogenous NO in a hypoxic area of cancer cells.

In an embodiment, the invention provides a method for a cancer therapy, the method comprising:
using experimental measurements to find ranges of Nitric Oxide (NO) concentrations and exposure durations which cause apoptosis of cancer cells residing in a hypoxic environment;
identifying an agent that can produce said ranges of Nitric Oxide (NO) concentrations and exposure durations, and
deploying the agent into a hypoxic area comprised by cancer cells,
   wherein the agent manipulates the concentration and duration of Nitric Oxide (NO) within said ranges to invoke apoptosis of the cancer cells.

### BRIEF DESCRIPTION OF THE DRAWINGS

The above and other aspects, features and advantages of the present invention will become more apparent upon consideration of the following description of preferred embodiments taken in conjunction with the accompanying drawing figures.

FIG. 1 provides a depiction of the double-edged sword effects of NO.

FIG. 2 depicts exemplary common nitric oxide targets in tumor biology.

FIG. 3 illustrates how exemplary Nitric Oxide (NO) biological effects are significantly different depending upon levels of NO concentrations [5].

FIG. 4A depicts a representative summary of some of the ideas leveraged by the invention along with a summary graph adapted from [12] relating to these.

FIG. 4B depicts a representative summary of some of the analysis results leveraged by the invention along with a summary Western blot graphic adapted from [12] relating to the above.

FIG. 5 depicts an exemplary representation of a central concept of the invention in the form of a 3-dimensional plot with vertical axis of NO level, horizontal axis of NO exposure duration, and axis into-the-page of cell survival time.

FIG. 6 depicts a first example experimental hypothesis can be used to establish if NO indeed regulates the expression of iNOS.

FIG. 7 depicts how the study and outcome of the second example hypothesis can be used to establish if NO indeed regulates the expression of iNOS.

FIG. 8 depicts how the study and outcome of a third example hypothesis can be used to establish if hypoxic environments within a cancer area affacts the expression of iNOS.

FIG. 9 depicts an example conceptual framework for experimental and therapy developing techniques as provided for by the invention.

FIG. 10A depicts a representation of the logical principle of an example NO quantitative kit, here the kit manufactured by Active Motif [32].

FIG. 10B depicts a representation of the detailed chemical reactions involved in the NO quantitative kit of FIG. 10A.

FIG. 11 depicts example cell culture incubation procedures with a selected NO donor.

FIG. 12A depicts an example of the type of releasing kinetic measurement data for the various NO donors that can be captured from this type of procedure.

FIG. 12B depicts an example data table capturing concentration values of NO from several kinds of NO donors at each time point in both the normal condition and hypoxic condition.

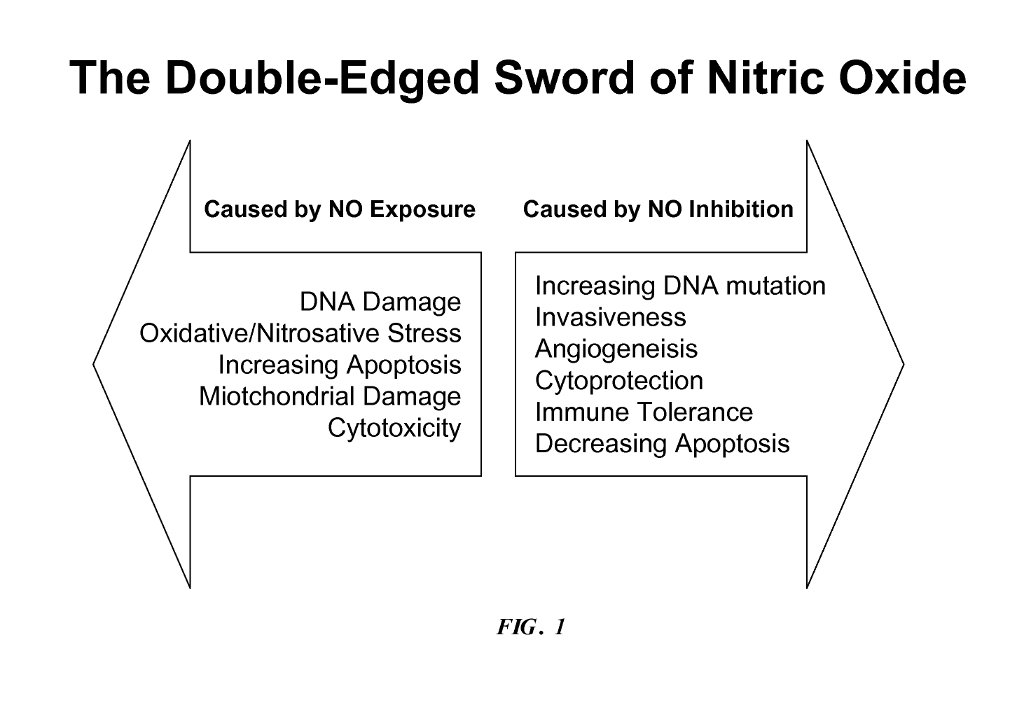

### DETAILED DESCRIPTION OF THE PREFERRED EMBODIMENTS

In the following, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

In the following description, reference is made to the accompanying drawing figures which form a part hereof, and which show by way of illustration specific embodiments of the invention. It is to be understood by those of ordinary skill in this technological field that other embodiments may be utilized, and structural, electrical, as well as procedural changes may be made without departing from the scope of the present invention.

Nitric Oxide (NO) and Cancer

Nitric oxide (NO) is small radical molecule that has ability to penetrate through the cells and regulated many pathways of biological functions and many physiological processes. NO has a short half-life in the body around 1-5 second [5]. At low concentration, NO serves as a signal transducer such as blood flow regulation, smooth muscle relaxation, iron homeostasis, platelet reactivity and neurotransmission. At high concentration levels, NO functions as cytotoxic defensive mechanism against pathogen and tumors. In addition, NO also plays roles in many pathological conditions such as inflammatory bowel disease, neurodegenerative disorders, and cancer [6].

NO can be produced by nitric oxide synthase (NOS) which has 3 isoforms: neuronal nitric oxide synthase (nNOS/NOSI), endothelium nitric oxide synthase (eNOS/NOSII), and inducible nitric oxide synthase (iNOS/NOSIII). Constitutive NOS, nNOS and eNOS, can be activated by calcium-calmodulin complex and the activity depends on concentration of calcium. However the activity of iNOS does not rely on the concentration of calcium. nNOS and eNOS regenerate nanomolar concentration of NO for durations on the order of second to minutes, while iNOS produces a nanomolar of NO durations on the order of hours to days [11]. Investigators report that NOS, especially iNOS, relates to tumor progression [5].

### Further Detail on the “Double-Edge Sword” Effects of NO

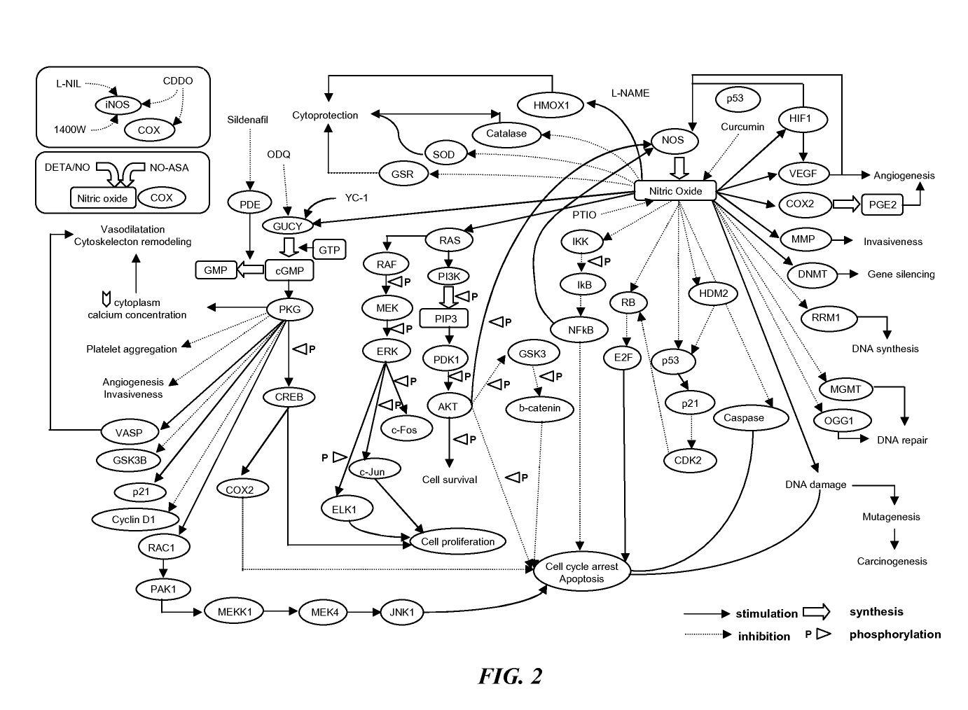

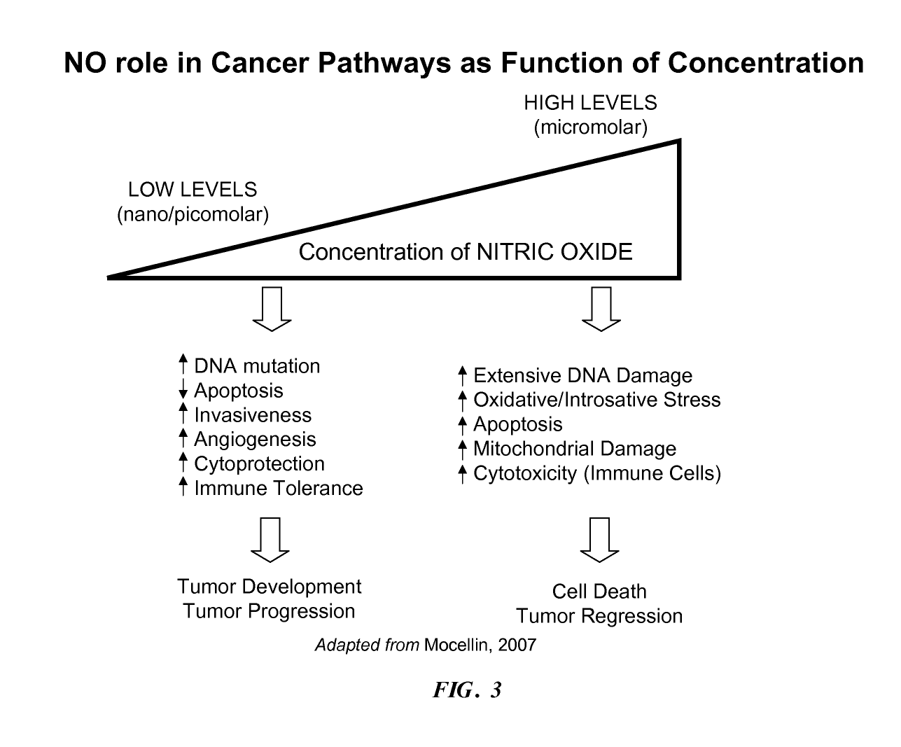

NO causes apoptosis by involving with many pathways such as p53, NFκB, MKP-1 and ERK. NO can activate the p53 pathway, which corresponds to DNA damage. NO inhibits the activity of transcriptional factor NFκB, antiapoptosis pathway. NO also induces the expression of MAPK phosphatase-1 (MKP-1) and ERK, causing cells commit suicide. On the other hand, NO prevents apoptosis due to in fact that it relates to several protein, ASK1, caspase, Ras and Akt. NO reduces the activity of caspase, which is a major apoptosis pathway. NO also inhibits the activity of Apoptosis Signal Regulating Kinase-1 (ASK1). Moreover, NO activate the antiapoptosis pathways such as Ras and Akt. In addition, NO promotes tumor growth by stimulating angiogenesis, related by stimulating of angiogenic factors such as VEGF, b-FGF and HIF-α. Another mechanism, by which NO might promote tumor cell growth is by stimulating the regeneration of prostaglandin, induces angiogenesis [5,12] as shown in FIG. 2, which depicts exemplary common nitric oxide targets in tumor biology [6]. NO plays role as a double-edged sword in the oncology by inhibiting and stimulating cancer progression. The effect of NO to cancer depends on the bioavailabilty of NO at the right time and right location [12]. FIG. 3 illustrates how exemplary Nitric oxide (NO) biological effects are significantly different depending upon levels of NO concentrations [5].

It is thought in current practice that the level of NO more likely relates to tumor progression and regression. Most investigators have showed that at low/intermediate steady-state concentration of NO (nano/picomolar) causes the progression of cancer by inducing DNA mutation, invasiveness, angiogenesis, cytoprotection and immune tolerance, while the high levels of NO (micromolar) cause cell apoptosis by inducing p53 phosphorylation, extensive DNA damage, oxidative/nitrosative stress, mitochondrial damage, cytotoxicity (again see FIG. 3) [13][14]. Therefore, the NO level is one of all parameters that should be concerned for therapeutic development of cancer disease.

Conventional radiotherapy is usually used for reoxygenation of tumors, which increases the efficiency of cytotoxic treatments. The evidences showed that the early tumor reoxygenation and vascular decomposition (tumor cell death) by ionizing radiation up-regulates eNOS expression, inducing vasodilation. However, the radiotherapy promotes tumor angiogenesis on the long-term (several days) [38]. Thus, sustaining NO production by continuing exposure to ionizing radiation might induce angiogenesis and tumor growth [39]. Accordingly, the duration of NO exposure is quite plausibly a key factor involved in tumor progression or regression.

The expression of iNOS was transcriptional up-regulated by pro-inflammatory cytokines, such as interferon (IFN), interleukin (IL-1, IL-2), tumor necrosis factor (TNF)-α, bacterial lipopolysaccharide, and hypoxia. iNOS gene was expressed by transcription factors, such as Nuclear Factor kappa-B (NFκB), JAnus Kinase Signal Transducer and Activator of Transcription (JAK/STAT), and c-Jun NH₂ terminal Kinase (JNK) [15]. In contrast, iNOS was transitionally down-regulated by steroid [16], anti-inflammatory cytokines, such as transforming growth factor- beta (TGF-β), IL-10 [16][17], p53 [18], and NO itself [19] (again as shown in FIG. 2). The negative feedback loop caused by the generating of NO induces p53 accumulation and down regulated iNOS expression [22, 23]. As a result, negative feedback of NO via iNOS can be cooperated to the effects of NO to tumor growth.

### Rationales Behind the Invention

Evidence has shown that NO induces apoptosis in a several cell types such as macrophages, neurons, pancreatic b-cells and thymocytes [41] while NO prevents apoptosis in some cell lines such as endothelial cells, lymphoma cells, ovarian follicles, cardiac myocytes and hepatocytes [34]. In same cell line, ovarian cells, on one hand, the investigators demonstrated that NO can cause apoptosis [37], on the other hand, other studies showed that NO associated to resistance to apoptosis [42]. In ovarian cancer cells studies, one presented that NO causes apoptosis by using nano-particle technology to control NO releasing, while in contrast, another report showed that NO prevents apoptosis did not control steady-state NO concentration. Moreover, responses of different cell types are involved in iNOS expression [42].

Accordingly, it is an inventive step to leverage the fact that the different effects of NO are caused from the different signal transduction and uncontrolled parameters such as the steady-state concentrations.

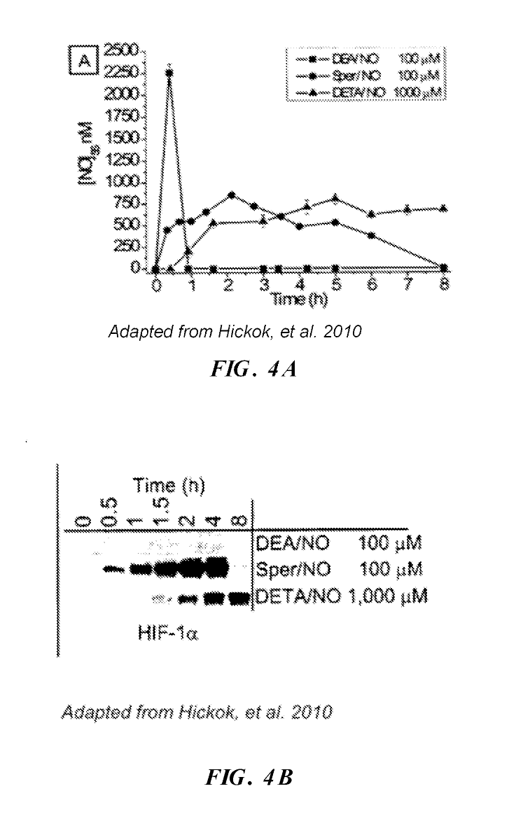

Investigation has shown that that NO causes HIF-α stabilization. In addition, the steady state of NO concentration depends on type of NO donors. FIG. 4A and FIG. 4B demonstrate that DEA/NO that produced high level of NO in media for only an hour did not regulate HIF-α expression, but DETA/NO and Sper/NO generated one-third the NO concentration as did DEA/NO, and provided this for several hours, resulting in stabilizing the HIF-α expression [27].

Accordingly, it is another inventive step to leverage the fact that the concentration of NO and duration of NO exposure can be estimated by regulated HIF-α expression.

Well-known publications have shown low concentration of NO (approximately 100-200 nm) is required for HIF-α stabilization, in contrast, the high levels of NO (more than 500 nm) stimulates p53 activation, causing apoptosis [12].

Accordingly, it is another inventive step to leverage the expected relation that the concentration and duration of NO exposure plays role in apoptosis pathways.

Moreover, it is another inventive step to leverage the expected relation that these factors might affect of iNOS as well.

Investigation has shown that NO caused the accumulation of p53, and p53 and iNOS were shown to participate in a negative regulatory loop [43]. However, Zhao S F., et al. demonstrated that p53 expression was elevated accompanying by the increased apoptotic cells but NO did not affect to iNOS expression [44]. Since the study from Shao using high concentration of NO donors did not show the effect of NO to iNOS, it is viable to assume that this could be due to high concentration of NO.

Accordingly, it is another inventive step to leverage the expected relation that high concentration of NO does not affect to iNOS expression but instead promotes cellular apoptosis.

NO regulates HIF-1 activation and angiogenesis. Due to the dependent effects of concentration of NO, the study has shown the metabolism of NO in hypoxia, low level of oxygen as in hypoxic tumor, is slower than the normal condition. Therefore, hypoxia will reduce the rate of local NO degradation and increase local NO concentration [12]. They also found that iNOS was down-regulated in adenocarcinomas [28]. Interestingly, malignant cancer cells are in hypoxic circumstance, but the many studies didn't control the level of oxygen in the experimental environment. For that reason, the results from these uncontrolled environment studies likely may not adequately answer or even address the appropriate questions.

Accordingly, it is another inventive step to leverage the expected relation that time of NO exposure under hypoxia will be longer than time of NO exposure under normoxia.

Further, it is another inventive step to leverage the expected relation that increasing time of NO exposure will cause down-regulation of iNOS by negative feedback effect.

Review of the aforementioned studies show that there are variants of the method of research and experiment that was not controlled, and it is fully viable that these caused the different effects of NO in either different cell lines or same cell lines.

Accordingly, it is another inventive step to leverage the expected relation that the factors that should be of concern are the concentration of NO, the duration of NO exposure, and the circumstance of NO exposure.

FIG. 4A provides a representative summary of some of the research findings leveraged by the invention, including:
Western blot results demonstrating the relationship between NO exposure and HIF-α accumulation in MCF-7.
NO caused the accumulation of p53, p53 and iNOS were shown to participate in a negative regulatory loop.
The metabolism of NO is slower at low oxygen concentration.
Each NO donor has different NO-releasing kinetic.
   FIG. 4A also provides a summary graph adapted from [12] relating to the above.

FIG. 4B provides a representative summary of some of the analysis results leveraged by the invention, including:
The results of [12] might stem from the NO down-regulate iNOS and low concentrations of NO causing up-regulation of HIF-α.
Hickok, et al [12] has showed NO didn't affect to iNOS expression, but it is noted that they studied at high conc of NO, μM.
Mostly, cancer cells exist in a hypoxic condition, but the studies of [12] did not adequately control the environment against a variety of known effects that are likely at play.
   FIG. 4B also provides a summary Western blot result adapted from [12] relating to the above.

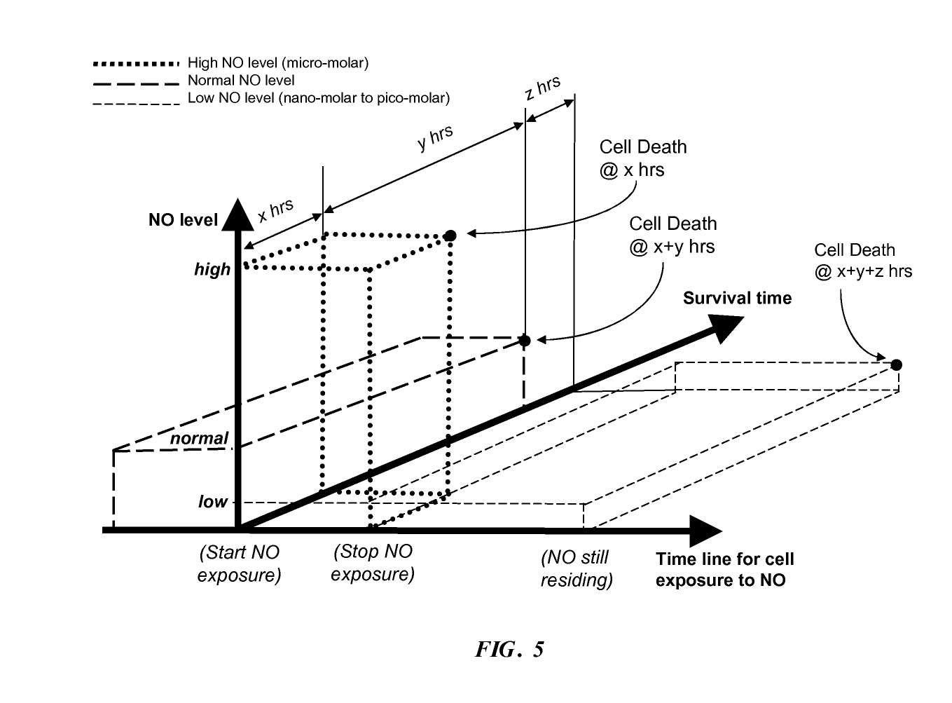

FIG. 5 depicts an exemplary representation of a central concept of the invention in the form of a 3-dimensional plot with vertical axis of NO level, horizontal axis of NO exposure duration, and axis into-the-page of cell survival time. The case depicted in the heavy-dashed line case represents the effects of a particular normal-range NO concentration which, when applied to normal cells, results in a cell lifetime of x+y hours. When starting the addition of a low-level (nano-molar to pico-molar concentration) exogenous NO, the cummulative NO level increases to an amount that cannot kill the cell, so this level causes negative feedback effect involving iNOS in the still living cell. iNOS, which was down-regulated from the the NO concentration of the previous case, will have less ability to produce NO; thus the cummulative NO concentration after stopping the addition of NO and associated long-duration NO exposure would promote normal cells to live longer , i.e., living for x+y+z hours, as shown in the light-dashed line case. In contrast, upon adding high (micro-molar)concentrations of NO large enough to enough induce apoptosis, this total concentration does not affect to iNOS expression and instead promotes earlier cell death at x hrs as shown in the bold dotted line case.

As described earlier, much evidence support the hypothesis that low NO concentration causes the progression of cancer cells, but high concentration of NO induce cancer cells apoptosis. However, to date there has not been any evidence demonstrating that factors that control the negative feedback of NO to iNOS expression can include NO concentration and durations of NO exposure.

Due to the fact that (1) iNOS participates in cancer progression and metastasis [12] and the fact that (2) one of the many pathways that regulate cancer progression is the negative feedback of NO to iNOS, it is viable to adopt the view that long term exposure of cancer cells to low concentration of exogenous NO might reduce iNOS expression by negative feedback effect. It is also viable to adopt the view that, in contrast, high concentration and short duration of NO exposure will not regulate iNOS expression but instead retard the survival of cancer cells.

Further, it is also viable to adopt the view that a low concentration of NO causes down-regulation of iNOS under hypoxia more than in normoxia. Since high level of oxygen in the environment, normoxic condition increases metabolism of NO.

The implications of understanding and leveraging the mechanisms controlling the balance between the NO related protective and destructive action could plausibly serve as a crucial modification of the regimen relating to exogenous NO in alternative tumor therapies.

### Experiment and Therapy Design

A key premise of the invention is that the concentrations of NO and duration of NO exposure regulates both iNOS expression and apoptosis. Here a research experiment design and accompanying methods are presented. The same steps can also be used in the design of a therapy.

In terms of experiment design, the central premise can be established and empirically explored in various ways. In one approach, a sequence of experimental hypotheses can be studied and confirms.

An example first hypothesis is that low concentration of exogenous nitric oxide causes negative feed back mechanism inducing down-regulation of iNOS and promoting tumor progression. As depicted in FIG. 6, the study and outcome of this first example hypothesis can be used to establish if NO regulates the expression of iNOS.

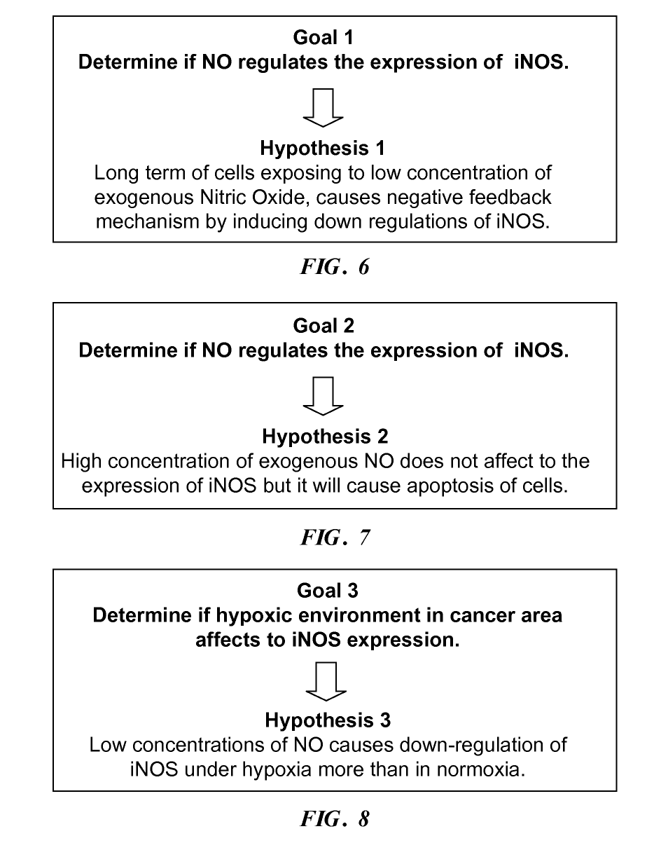

An example second hypothesis is that high concentration of exogenous NO does not affect to the expression of iNOS but it will cause apoptosis of cells. As depicted in FIG. 7, the study and outcome of this second example hypothesis can also be used to establish whether or not that NO indeed regulates the expression of iNOS.

An example third hypothesis is that low concentration of NO causes down-regulation of iNOS is expected to be more highly demonstrated under hypoxia conditions rather than under normoxia conditions. An experimental program for the example third hypothesis could, for example, study the limit of each parameter that can affect the outcome. In a plausable situation, such a study can be used to improve the hypothesis. Additionally, since it is well-known that NO donors have diverse releasing kinetics, an experimental program for the example third hypothesis could study the kinetics of NO releasing for each of the candidate NO donors so as to identify those select NO donor(s) that have kinetics suitable for study of the example third hypothesis. As depicted in FIG. 7, the study and outcome of this third example hypothesis can be used to establish if hypoxic environments within a cancer area affects the expression of iNOS.

In an inventive step, the above experimental procedures can be combined with candidate therapeutic agents that are designed to produce conditions matching those of particular situations that appear advantageous for control of cancer processes.

### Experimental Methods

One way to test for the expression of iNOS is to use the Western blot method. Here the expression of iNOS can be determined by titrating several concentration of NO from 25 nM to 1 μM. At the same time, the response of iNOS to controlled variations in the duration of NO exposure of cancer cell can be determined. More specifically, the survival of cancer cells incubated in several concentration levels of NO and several time of NO exposure.

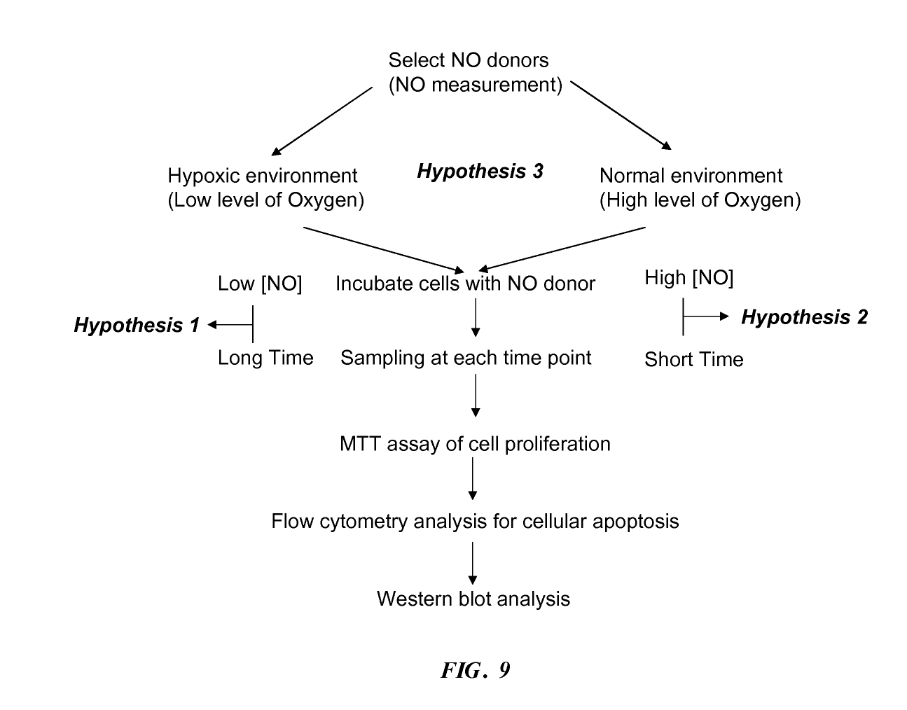

FIG. 9 depicts an example conceptual framework for experimental and therapy developing techniques as provided for by the invention. Methodological considerations and design innovations depicted therein include:
Approaches to the selection of cancer cell line to be used;
Approaches to Nitric Oxide measurement;
Approaches to use of MTT assay;
Approaches to use of Western blot analysis.
   Each of these is considered in turn in the material that follows.

### Example Selection Cancer Cell Line

Since there are many different types of cancer, it is entirely possible that there could be diversity in the regulation of the NO and iNOS pathways. In addition, the effect of NO depends on not only its concentration, but it also relies on type of reaction pathway in the cell [33]. As NO is a double-edge sword, NO can prevent apoptosis in some cell types such as endothelial cells, lymphoma cells, ovarian follicles [36], cardiac myocytes, and hepatocytes [34], while promote apoptosis in some cell types, for example cell types including macrophages, neurons, pancreatic b-cells, thymocytes , and chondrocytes [35]. However, one study shows that improving the NO delivery to the target cells causes the apoptosis of healthy ovarian cells. In an example experiment design for testing the hypotheses, one can therefore, as an example, study the dependence of concentration NO and duration of NO exposure on iNOS expression from ovarian cancer cell lines so as to leverage the opposing results from the aforementioned studies of NO effect on ovarian cells. Other approaches can be used of course, and these are anticipated by the invention.

### Nitric Oxide Measurement

Because NO has a short half-life, the concentration of NO and the half-life of NO depends on type of NO donor. In an example experiment design for testing hypotheses, one can reference and/or measure the half life of NO from each NO donor. Additionally, since the metabolism of NO at normal and hypoxic conditions are different, an example experiment design for testing hypotheses can include study of the regulation of concentration NO and duration of NO exposure in at least normal and hypoxia conditions.

In more detail, NO donors can play an important role in cancer therapy because the NO donors are the agents generating and releasing exogenous NO into the cancer area environment. In general, various NO donor compounds differ in at least one of the amounts of NO produced, NO production half-life, and release kinetics. In addition, the microarray studies showed the same cell line using different NO donors provide different experimental results.

Thus an example experiment design for testing hypotheses can include the factors of NO concentration and the half-life of exogenous NO from NO donors [12]. Such an experiment design can include measurement of the concentration of NO for each NO donor every 30 minutes until the measurable concentration of NO disappears. The results from such an experiment can be used to identify candidate NO donor compounds suitable for continued research and therapeutic applications.

Not only do the NO releasing kinetics vary with the kind of NO donor, but the NO releasing kinetics also varies in response to other aspects of the chemical environment, for example such as the presence of oxygen. In normal conditions, high levels of oxygen in the environment cause NO to be metabolized faster than in a hypoxia (poor oxygen) condition. Thus an example experiment design for testing hypotheses can include conditions with low levels of oxygen. For example, an example experiment design for testing hypotheses can include study of the half-life of NO in normal and hypoxia condition.

### Nitric Oxide Donor Selection

Regarding NO donor selection, different types of NO donors are separately exclusively relevant to the different hypothesis or therapy design steps. For example:

Hypothesis 1: This hypothesis states that long term exposure of cells to low concentration of exogenous NO causes negative feedback mechanism to induce down-regulation of iNOS. To confirm this hypothesis, one can choose NO donor that can produce low levels of NO and which has a long half-life. In one approach, that NO donor can be applied to cell culture media at every time point in the experiment, and the levels of iNOS and NO are monitored and recorded. One example test outcome would include data useful to calculate the conditions related to maintaining a steady NO level.

Hypothesis 2: This hypothesis states that high concentration of exogenous NO does not affect to the expression of iNOS but instead will cause apoptosis of cells. To prove this hypothesis, one can choose a NO donor that can produce high level of NO but which has short half-life. In one approach, cell sampling can be used to check iNOS expression and cell apoptosis.

Hypothesis 3: This hypothesis states that for a given level of NO, the expression of iNOS down-regulates more under hypoxic conditions than in normal (richer oxygen) conditions. To prove this hypothesis, one can choose a NO donor generating low NO concentration and compare iNOS response between normal and hypoxic environment.

### Quantitative NO Measurement Kit Technology

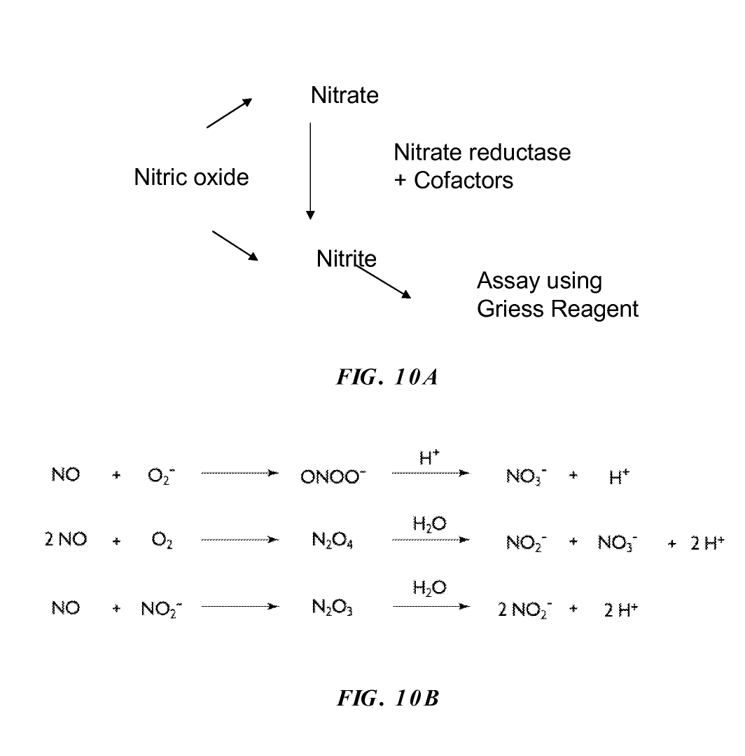

Due to the fact that NO is unstable in the applicable environment, and more specifically has a short half-life therein, and further the response of iNOS expression to the concentration of NO during a period of time is an important measurement goal, provisions for accurate quantitative NO measurement is of significant importance. As to this, note for example that NO releasing can be measured by observing the converse of NO to nitrite and nitrate in a tissue culture media. Such a measurement method is commercial available in the form of a NO quantitation kit, for example from Cayman Chemical. Alternatively NO quantitation kits employing cofactor technologies (as in the kit from Active Motif) are also commercially available, FIG. 10A depicts a representation of the logical principle of an example NO quantitative kit, here the kit manufactured by Active Motif [32].

FIG. 10B depicts a representation of the detailed chemical reactions involved in the NO quantitative kit of FIG. 10A. NO contained in the sample is metabolized to nitrate and nitrite, and then nitrate is converted to nitrite by nitrate reductase. The nitrite will react with Griess reagent, azo compound, which turns the color to be purple and it can be detected by spectrophotometer. In this fashion (or variations upon it), one can determine the concentration NO from NO nitrite metabolite. It is noted, however, that NADPH (a crucial cofactor for nitrate reductase) decreases the sensitivity of this method by interfering the Griess reaction. To increase the sensitivity, lactate dehydrogenase (LDH) can be added to the step prior to Griess reaction (which leverages the NADPH scavenge ability).

### Western Blot Technology

Protein blotting, also known as western blotting and Edman sequencing, is analytical method that relates to immobilization of protein. This method measures the relative amount of protein and analyzes the results of co-immonoprecipitation experiments. This technique use polyvinylidene difluoride (PVDF) membrane and apply the monoclonal or polyclonal antibodies before detection. SDS—polyacylamide gel eletrophoresis (SDS-PAGE) is used for separating protein from different molecular weight and isoforms. The target protein is bound with the specific antibody in the sample and detected by chemiluminescence. The protein is transferred to the PVDF membrane by electroblotting and the membrane provides the information of protein sequencing [29,30,31].

### Additional Example Materials, Cell Lines, Instruments, and Procedure Details

The main reagents of consideration are NO donor compounds. Examples of these include DEA/NO, DETA/NO, Sper/NO, PABA/NO, SNP, GTN, SNAP, and GSNO. This collection, in fact, contains the range of NO production level and half-life characteristics that would be required across the various hypotheses and their corresponding therapy design steps.

Example antibodies for this experiment are iNOS rabbit poly-clonal and IgG 1:1500. MTT reagent can be used to analyze for cellular proliferation, for example employing a microtiter-plate reader.

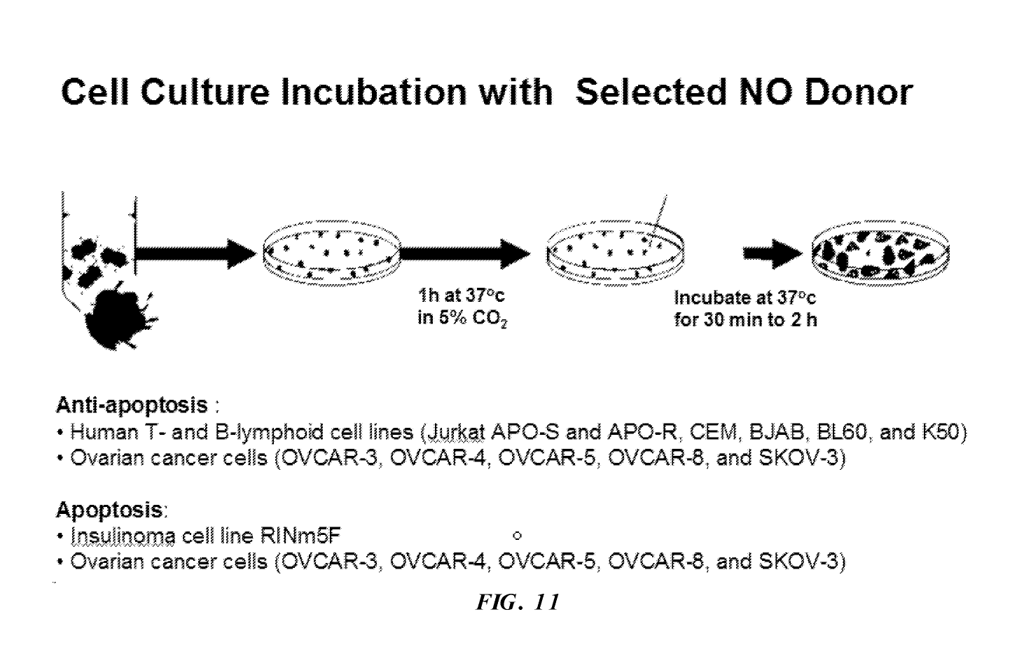

As mentioned earlier, a prime candidate for cancer cell lines are ovarian cancer cells, for example OVCAR-3. Ovarian cancer cell lines (for example OVCAR-3) can be cultured in the media before use for all of the experiment or therapy design. FIG. 11 depicts example cell culture incubation procedures with a selected NO donor.

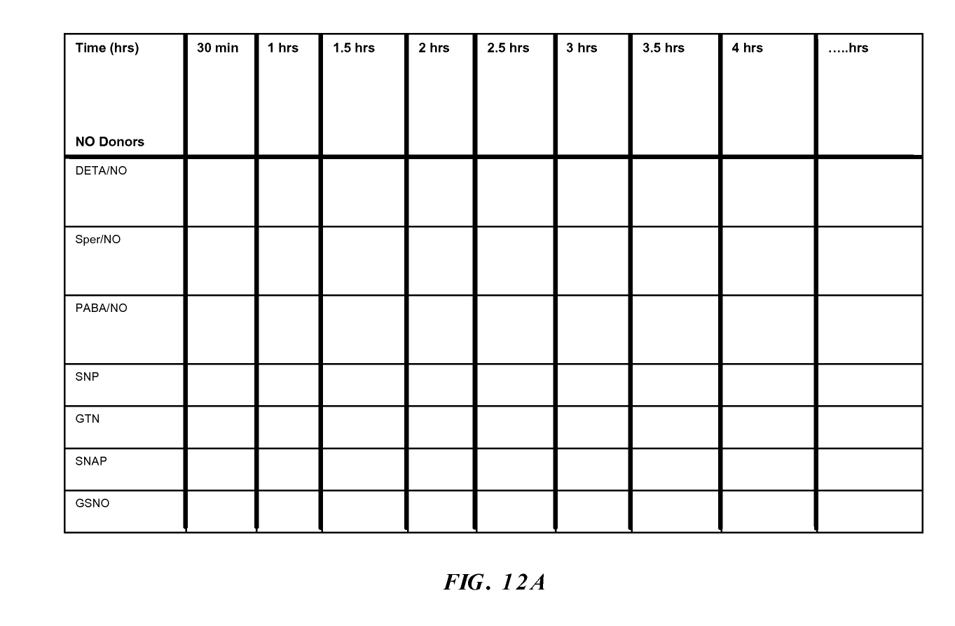

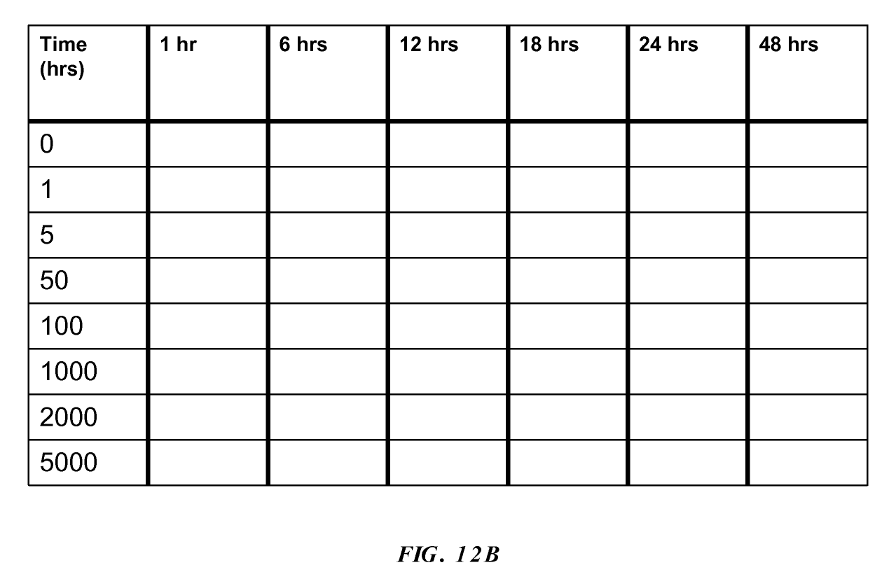

The releasing kinetic for the various NO donors (for example, DEA/NO, DETA/NO, Sper/NO, PABA/NO, SNP, GTN, SNAP, and GSNO) can be studied the in hypoxic and normal condition by using NO quantitative kit. FIG. 12A depicts an example of the type of releasing kinetic measurement data for the various NO donors that can be captured from this type of procedure. For example here, after choosing the right NO donor for testing each hypothesis, a NO kit will be used for study the NO production quantity and NO production half-life of the NO donor at each concentration in hypoxic and normal environments. Again results of this test or therapy design step can address how much NO donor can be added to the culture media to get a desired total concentration of NO and when the NO donor can be added to the culture media to maintain the steady state NO level.

MTT Assay for measurement of Cellular Proliferation

The effects of NO on apoptosis can be measured via cell proliferation colorimetric assays using enzyme processable dyes such as MMT (3-(4,5-Dimethylthiazol-2-yl-2,5-diphenyltetrazolium bromide, a yellow tetrazole), XTT, MTS, WSTs or other dyes that naturally exhibit a first color (such as the yellow of MTT) and reduce by living cells to a dye exhibiting a different color (such as the purple of formazan dyes).

To study the cellular proliferation of cancer cell lines, each type of cancer cell line will be incubated with selective NO donor at the concentration that will release steady optimal concentration. FIG. 12B depicts an example data table capturing concentration values of NO from several kinds of NO donors at each time point in both the normal condition and hypoxic condition. The cell lines will be sampled at each time point so as to study cellular proliferation by using MTT reagent. The inhibition rate (RT) of the selective NO donor can then be calculated, for example, from an absorbance reading taken using a microtiter plate reader as stated by the following formula:

% IR=(1-ODₑₓₚₑᵣᵢₘₑₙₜ/OD_{control})×100

The results of such an experiment or therapy development step can identify when the culture cells have the highest % IR (i.e., what time point that NO will start inhibiting cellular proliferation) and what concentration will cause the maximum of % IR (i.e., the concentration level causing cell death). In addition, the results such an experiment or therapy development step can identify the range of NO concentration and length of time that the culture cells can survive in those circumstances.

Western Blot Analysis for Expression of iNOS

At each concentration and sampled time point of an NO donor's incubation, the cancer cell lines can be corrected for testing the expression of iNOS. The incubated cells will be broken through use of a cell lysis procedure to extract protein. The protein can then be electrophoresed, for example, on a 10% SDS-PAGE gel electrophoresis system and transferred to nitrocellulose membranes. The membrane can then, for example, be incubated overnight with a primary antibody to iNOS, and then incubated with the second antibody for 1 hour. The iNOS can then be detected, for example, by an enhanced chemiluminescence system.

The results of such experiments or therapy development steps can demonstrate what concentration of NO and which time point of incubation that NO caused a down-regulation of iNOS. Additionally, such an experiment or therapy development step can identify what time point and what concentration of NO will not have an effect on iNOS expression.

The table of FIG. 12B also depicts exemplary sampling planning of NO cellular exposure using, for example MTT assay, Western blot methods, and flow cytometry analysis.

### Creating Cancer Therapies Through Use of the Aforedescribed Data and Methods of The Invention

The invention provides for various aspects of the experiment data and experiment design to be used in creating a cancer therapy that manipulates exogenous NO in hypoxic areas of cancer cells.

In one approach, an agent comprised by the cancer therapy is used to manipulate manipulates exogenous NO in hypoxic areas of cancer cells so as to invoke apoptosis in those of cancer cells.

In one approach, an agent comprised by the cancer therapy is used to replace at least one NO donor in at least one portion of at least one of the procedures described.

In an approach to creating the cancer therapy, an agent comprised by the cancer therapy controls the level of NO concentration.

In an approach to creating the cancer therapy, an agent comprised by the cancer therapy controls the duration of NO exposure.

Additionally, the invention provides for use of the aforedescribed methods to create a cancer therapy. The experimental methods and measurements produced by them are used to identify ranges of NO concentrations and NO exposure durations which cause apoptosis of cancer cells residing in a hypoxic environment. Using this, one or more agents are identified that can produce these ranges of NO concentrations and NO exposure durations. The therapy would then comprise deploying the agent into a hypoxic area comprised by cancer cells wherein the agent manipulates the NO concentration and NO exposure duration within said ranges, resulting in apoptosis of the cancer cells,

In an example embodiment or implementation of such a cancer therapy, the agent comprises at least one NO donor.

The terms “certain embodiments”, “an embodiment”, “embodiment”, “embodiments”, “the embodiment”, “the embodiments”, “one or more embodiments”, “some embodiments”, and “one embodiment” mean one or more (but not all) embodiments unless expressly specified otherwise. The terms “including”, “comprising”, “having” and variations thereof mean “including but not limited to”, unless expressly specified otherwise. The enumerated listing of items does not imply that any or all of the items are mutually exclusive, unless expressly specified otherwise. The terms “a”, “an” and “the” mean “one or more”, unless expressly specified otherwise.

While the invention has been described in detail with reference to disclosed embodiments, various modifications within the scope of the invention will be apparent to those of ordinary skill in this technological field. It is to be appreciated that features described with respect to one embodiment typically can be applied to other embodiments.

The invention can be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

Although exemplary embodiments have been provided in detail, various changes, substitutions and alternations could be made thereto without departing from spirit and scope of the disclosed subject matter as defined by the appended claims. Variations described for the embodiments may be realized in any combination desirable for each particular application. Thus particular limitations and embodiment enhancements described herein, which may have particular advantages to a particular application, need not be used for all applications. Also, not all limitations need be implemented in methods, systems, and apparatuses including one or more concepts described with relation to the provided embodiments. Therefore, the invention properly is to be construed with reference to the claims.

### REFERENCES

[1] Herr K., Debatin M., 2001. Cellular stress response and apoptosis in cancer therapy, Blood, 98, 2603-2614.

[2] Louis J. Ignarro. 2000. Nitric Oxide: Biology and Pathobilogy (Second Edition), Academic Press, 813-830.

[3] Bonovida B., Baritak S., Huerta Yepes S., Vega M I., Chatterjee D., Yeung K., 2008. Novel therapeutic application of nitric oxide donors in cancer: roles in chemo-and immono sensitization to appotosis and inhibition of metastases, Nitric Oxide, 19, 152-157.

[4] Huerta S., Chilka S., Bonovida B., 2008. Nitric Oxide donors: Novel cancer therapeutics (review). Int. J. Oncol, 33, 909-927.

[5] Mocellin S., Bronte V., Nitti D, 2007. Nitric Oxide, a double edged sword in cancer biology: searching for therapeutic opportunities, Medicinal Research Reviews, 27.3, 317-352.

[6] Mocellin, S. 2009. Nitric oxide: cancer target or anticancer agent?, Current Cancer Drug Targets, 9, 214-228.

[7] Kim, Y M., Kim T H., Chung H T, Talanian R V., Yin X M., Billiar T R., 2000. Nitric oxide prevents tumor necrosis factor alphainduced rat hepatocyte apoptosis by the interruption of mitochondrial apoptotic signaling through S-nitrosylation of caspase-8. Hepatology, 32, 770-778.

[8] Park H S., Yu J W., Cho J H., Kim M S., Huh S H., Ryoo K., Choi E J., 2004. Inhibition of apoptosis signal-regulating kinase 1 by nitric oxide through a thiol redox mechanism. J. Biol. Chem., 279, 7584-7590.

[9] Marshall H E., Stamler J. S. 2001. Inhibition of NF-kappa B by Snitrosylation, Biochemistry, 40, 1688-1693.

[10] Brune B., 2003. Nitric oxide: NO apoptosis or turning it ON? Cell Death Differ, 10, 864-869.

[11] Michel T., Feron O., 1997. Nitric oxide synthases: which, where, how, and why? J. Clin. Invest., 100, 2146-2152.

[12] Hickok J R., Thomas D D., 2010. Nitric Oxide and cancer therapy: the emperor has NO cloths, Current Pharaceutical Design, 16, 381-391.

[13] Postovit L M., Adams M A., Lash G E., Heaton J P., Graham C H., 2004. Nitric oxide-mediated regulation of hypoxia induced B16F10 melanoma metastasis, Int. J. Cancer, 108(1), 47-53.

[14] Dhar A., Brindley J M., Stark C., Citro M L., Keefer L K., Colburn N H., 2003. Nitric oxide does not mediate but inhibits transformation and tumor phenotype. Mol Cancer Ther, 2(12), 1285-1293.

[15] Ganster R W., Taylor B S., Shao L., Geller D A., 2001. Complex regulation of human inducible nitric oxide synthase gene transcription by Stat 1 and NF-kappa B. Proc. Natl. Acad. Sci. USA, 98, 8638-8643.

[16] Shinoda J., McLaughlin K E., Bell H S., Swaroop G R., Yamaguchi S., Holmes M C., Whittle I R., 2003. Molecular mechanisms underlying dexamethasone inhibition of iNOS expression and activity in C6 glioma cells. Glia, 42, 68-76.

[17] Wink D A., Vodovotz Y., Laval J., Laval F., Dewhirst M W., Mitchell J B., 1998. The multifaceted roles of nitric oxide in cancer, Carcinogenesis, 19, 711-721.

[18] Chen Y H., Layne M D., Chung S W., Ejima K., Baron R M., Yet S F., Perrella M A., 2003. Elk-3 is a transcriptional repressor of nitric-oxide synthase 2. J. Biol. Chem., 278, 39572-39577.

[19] Ambs S., Ogunfusika M O., Merriam W G., Bennett W P., Billiar T R., Harris C C., 1998. Up-regulation of inducible nitric oxide synthase expression in cancer-prone p53 knockout mice. Proc. Natl. Acad. Sci. USA, 95, 8823-8828.

[20] Norbury C J., Zhivotovsky B., 2004. DNA damage-induced apoptosis, Oncogene, 23, 2797-2808.

[21] Hussain S P., Hofseth L J., Harris C C., 2003. Radical causes of cancer, Nat. Rev. Cancer, 3, 276-285.

[22] Brune B., 2003. Nitric oxide: NO apoptosis or turning it ON? Cell Death Differ., 10, 864-869.

[23] Ambs S., Ogunfusika M O., Merriam W G., Bennett W P., Billiar T R., Harris C C., 1998. Up-regulation of inducible nitric oxide synthase expression in cancer-prone p53 knockout mice. Proc. Natl. Acad. Sci. USA 95, 8823-8.

[24] Wink D A., Vodovotz Y., Laval J., Laval F., Dewhirst M W., Mitchell J B., 1998. The multifaceted roles of nitric oxide in cancer, Carcinogenesis, 19, 711-721.

[25] Chen Y H., Layne M D., Chung S W., Ejima K., Baron R M., Yet S F., Perrella M A., 2003. Elk-3 is a transcriptional repressor of nitric-oxide synthase 2, J. Biol. Chem., 278, 39572-39577.

[26] Hinz B., Brune K., Pahl A., 2000. Nitric oxide inhibits inducible nitric oxide synthase mRNA expression in RAW 264.7 macrophages, Biochem. Biophys. Res. Commun., 271, 353-357.

[27] Thomas D D., Espey M G., Ridnour L A., Hofseth L J., Mancardi D., Harris C C., 2004. Hypoxic inducible factor 1 alpha, extracelluar signal-regulated kinase, and p53 are regulated by distinct threshold concentration of nitric oxide. Proc. Natl. Acad. Sci. USA, 101: 8894-9.

[28] Ramaswamy S., Riss J N., Lander E S., 2003. Gikyb/tR A molecular signature of metastasis in primary solid tumors. Nat. Genet., 33, 49-54.

[29] http://www.westernblotting.org/

[30] Penna A., Cahalan M., 2007. Western Blotting Using the Invitrogen NuPage Novex Bis Tris MiniGels. JoVE. 7.

[31] Komatsu S., 2009. Western blotting/Edman sequencing using PVDF membrane, Method in molecular biology, 536,163-170.

[32] www.activemotif.com.

[33] Brune B., 2005. The intimate relation between nitric oxide and superoxide in apoptosis and cell survival. Antioxide. Redox Signal, 7, 497-507.

[34] Frederick L. K., Zhang X., 2002. Apoptosis: biochemical aspects and clinical implications, Clinica Chimica Acta, 326, 27-45.

[35] Melino G., Catani M V., Corazzari M, Guerrieri P., Bernassola F., 2000. Nitric oxide can inhibit apoptosis or switch it into necrosis, Cell. Mol. Life Sci. 57, 612-622.

[36] Chun S Y., Eisenhauer K M., Kubo M., 1995. Interleukin-1h suppresses apoptosis in rat ovarian follicles by increasing nitric oxide production. Endocrinology, 136, 3120-3127.

[37] Ellen V S., Alexis W C., Shin J H., Liu J., Der C J., Schoenfisch M H., 2010. Nitric Oxide-Releasing Silica Nanoparticle Inhibition of Ovarian Cancer Cell Growth, Mol. Pharmaceutics, Article ASAP.

[38] Sonveaux P., Brouet A., Havaux X., 2003. Irradiation-induced angiogenesis through the up-regulation of the nitric oxide pathway: implications for tumor radiotherapy. Cancer Res., 63(5), 1012-1019.

[39] Sonveauxa P., Jordanb B F., Gallezb B., Feron O., 2009. Nitric oxide delivery to cancer: Why and how?, EUROPEAN JOURNAL OF CANCER, 45, 1352-1369.

[40] Brune B, Zhou J., 2007. Nitric oxide and superoxide: interference with hypoxic signaling. Cardiovasc. Res., 75(2), 275-82.

[41] Melino G., Catani M., Corazzari V., M., Guerrieri P and Bernassola F., 2000. Nitric oxide can inhibit apoptosis or switch it into necrosis, CMLS, Cell. Mol. Life Sci. 57, 612-622.

[42] Nomelini S R., Ribeiro C A., Tavares-Murta B M., Adad S J., Candido Murta E F., 2008. Production of Nitric Oxide and Expression of Inducible Nitric Oxide Synthase in Ovarian Cystic Tumors, Mediators Inflamm. 186584.

[43] Gallo O., Schiavone N., Papucci L., Sardi I., Magnelli L., Franchi A., Masini E., Capaccioli S., 2003. Down-regulation of nitric oxide synthase-2 and cyclooxygenase-2 pathways by p53 in squamous cell carcinoma. Am. J. Pathol., 163, 723-732.

[44] Zhao S F., Tong X. Y., Zhu F D., 2005. Nitric oxide induces oral squamous cell carcinoma cells apoptosis with p53 accumulation, Oral Oncology , 41, 785-790.

I claim:

1. A method for designing experiments for the study of Nitric Oxide (NO) in the control of cell death for use in cancer therapies, the method comprising:
establishing that long term exposure of cells to low concentration of exogenous nitric oxide causes a negative feedback mechanism to induce down-regulation of inducible Nitric Oxide Synthase (iNOS) by choosing a first NO donor that can produce a relatively low level of NO concentration over a relatively long NO-production half-life, applying that NO donor to a cell culture media at selected time points, measuring the levels of iNOS concentration and NO concentration to produce first measurements, and recording first data corresponding to the first measurements;
establishing that high concentration of exogenous NO does not affect to the expression of iNOS but instead will cause apoptosis of cells by choosing a second NO donor that can produce a relatively high level of NO concentration over a relatively short NO-production half-life, applying that NO donor to a cell culture media at selected time points, measuring the levels of iNOS and NO concentration to produce second measurements, and recording second data corresponding to the second measurements; and
establishing that for a given level of NO concentration, the expression of iNOS down-regulates more in hypoxic conditions than in normal oxygen conditions by choosing a third NO donor that generates a relatively low concentration of NO, applying that NO donor to a cell culture media at selected time points, measuring the levels of iNOS and NO concentration to produce third measurements, recording third data corresponding to the third measurements, and comparing the iNOS response between normal and hypoxic environments,
wherein at least one of the first data, second data, and third data are used to create a cancer therapy that manipulates exogenous NO in a hypoxic area of cancer cells.

2. The method of claim 1 wherein an agent comprised by the cancer therapy is used to replace at least one NO donor in at least one portion of the method.

3. The method of claim 1 wherein cell sampling is used to measure iNOS expression.

4. The method of claim 1 wherein cell sampling is used to measure cell apoptosis.

5. The method of claim 1 wherein cellular proliferation by using colorimetric assay.

6. The method of claim 5 wherein the colorimetric assay uses MTT dye.

7. The method of claim 5 wherein the colorimetric assay uses XTT dye.

8. The method of claim 5 wherein the colorimetric assay uses MTS dye.

9. The method of claim 5 wherein the colorimetric assay uses WSTs dye.

10. The method of claim 1 wherein Western blot analysis is used to measure iNOS expression.

11. The method of claim 1 wherein the cell culture media comprises ovarian cancer cells.

12. The method of claim 1 wherein an agent comprised by the cancer therapy is used to replace at least one of the first, second, and third NO donor.

13. The method of claim 1 wherein an agent comprised by the cancer therapy controls the level of NO concentration in the hypoxic area comprised of cells.

14. The method of claim 1 wherein an agent comprised by the cancer therapy controls the duration of NO exposure in the hypoxic area comprised by cancer cells.

15. A method for designing a cancer therapy by manipulating the concentration and duration of Nitric Oxide (NO) for the control of cell death, the method comprising:
using a first NO donor to produce a relatively low level of NO concentration over a relatively long NO-production half-life, applying that NO donor to a cell culture media at selected time points, measuring the levels of inducible Nitric Oxide Synthase (iNOS) and NO concentration to produce first measurements, and recording first data corresponding to the first measurements;
using a second NO donor to produce a relatively high level of NO concentration over a relatively short NO-production half-life, applying that NO donor to a cell culture media at selected time points, measuring the levels of iNOS and NO concentration to produce second measurements, and recording second data corresponding to the second measurements; and
using a third NO donor to produce a relatively low concentration of NO, applying that NO donor to a cell culture media at selected time points, measuring the levels of iNOS and NO concentration to produce third measurements, recording third data corresponding to the third measurements, and comparing the iNOS response between normal and hypoxic environments,
wherein at least one of the first data, second data, and third data are used to create a cancer therapy that manipulates exogenous NO in a hypoxic area of cancer cells.

16. The method of claim 15 wherein the cancer therapy comprises an agent that manipulates exogenous NO in the hypoxic area of cancer cells.

17. The method of claim 16 wherein the agent comprises at least one NO donor.

18. A method for a cancer therapy, the method comprising:
using experimental measurements to find ranges of Nitric Oxide (NO) concentrations and exposure durations which cause apoptosis of cancer cells residing in a hypoxic environment;
identifying an agent that can produce said ranges of Nitric Oxide (NO) concentrations and exposure durations, and
deploying the agent into a hypoxic area comprised by cancer cells,
wherein the agent manipulates the concentration and duration of Nitric Oxide (NO) within said ranges to invoke apoptosis of the cancer cells.

19. The method of claim 19 wherein the agent comprises at least one NO donor.
